(19) United States
(12) Patent Application Publication
(10) Pub. No.: US 2026/0263342 A1
(43) Pub. Date: Sep. 10, 2026

(54) NEW SOLID COSMETIC COMPOSITIONS AND USE THEREOF FOR HYGIENE OF THE SKIN OR HAIR

(71) Applicant: PIERRE FABRE DERMO-COSMETIQUE, LAVAUR (FR)

(72) Inventors: Emmanuelle GUILLET, CASTRES (FR); Jean-Luc BLETTERY, CASTRES (FR); Anne-Laure GAUDRY, CASTRES (FR)

(73) Assignee: PIERRE FABRE DERMO-COSMETIQUE, LAVAUR (FR)

(21) Appl. No.: 19/165,567

(22) PCT Filed: Mar. 13, 2024
(86) PCT No.: PCT/EP2024/056603

§ 371 Date: Sep. 15, 2025

(30) Foreign Application Priority Data
Mar. 16, 2023 (FR) FR2302435

(51) Int. Cl. A61K 8/46; A61K 8/02; A61K 8/44; A61K 8/60; A61Q 19/10

(52) U.S. Cl. CPC A61K 8/466; A61K 8/0229; A61K 8/442; A61K 8/602; A61Q 19/10

(57) ABSTRACT

The invention relates to a solid cosmetic composition which is particularly suitable for the care and/or hygiene of the skin and/or hair. This composition contains 30 to 65% by weight of consistency agent(s), a little water, at least one nonionic surfactant chosen from alkyl glucosides in which the alkyl radical comprises 8 to 16 carbon atoms, and at least one anionic surfactant chosen from isethionic acids and the cosmetically acceptable salts thereof, in a total content of less than or equal to 20% by weight. This composition has a high degree of naturalness and good foamability.

The present invention relates to a cosmetic composition in solid form and to the use of such a composition for washing and/or caring for the skin and/or hair. The invention also relates to a cosmetic treatment method for skin and/or hair hygiene and/or care using such a composition.

### PRIOR ART

The market for solid cosmetics is currently trending, and many companies commercialize compositions in the solid form, which are more environmentally friendly than liquid compositions, for washing and/or caring for the body, face or hair.

The oldest solid cosmetic compositions, widely used by consumers, are soaps, products that are obtained by a saponification reaction. However, due to their high pH, typically greater than 10, soaps are very aggressive to the skin, particularly sensitive skin, and are not suitable for use on hair.

More recently, syndets, synthetic detergents, have emerged as advantageous alternatives to the products produced by saponification. Syndets have the same cleansing properties as conventional soaps, including good foaming properties, but are less harsh on the skin.

Examples of products proposed by the prior art that are based on such syndets include the “Shampoo bar” product of the company Hopery Benjamin Böhme (Mintel datasheet 8088547), sold on the German market, and the “Solid Shampoo” product of the company L'Occitane (Mintel datasheet 10325914), sold on the Belgian market.

The list of ingredients given on the packaging of these products shows in first position, respectively, for the first product, sodium cocoyl isethionate and, for the second product, disodium lauryl sulfosuccinate followed immediately by sodium cocoyl isethionate. In accordance with Article 19 1 g), paragraph 4, of (EC) Regulation No. 1223/2009 of the European Parliament and Council of Nov. 30, 2009 on cosmetic products, the list of ingredients mentioned on the packaging must be given in descending order of weight at the time of incorporation into the cosmetic product. It is thus understood that sodium cocoyl isethionate is the main ingredient of the first of these formulations, and disodium lauryl sulfosuccinate and sodium cocoyl isethionate are the main ingredients of the second of these formulations. However, consumers are becoming increasingly vigilant about the composition of their cosmetic products, and are looking for products that include few chemical ingredients, such as isethionates and sulfosuccinates, to which they prefer ingredients of natural origin, known for their greater tolerance and affinity with the skin, and which are more environmentally friendly. However, the quantity and/or quality of foam produced by products based on natural ingredients are generally low.

Developing cosmetic compositions that have both a high degree of naturalness and very good foaming ability is a challenge, particularly since the combination of these properties preferably has to be accompanied by other highly desirable characteristics for such cosmetic compositions, in particular with regard to their consistency, stickiness, appearance and sensory characteristics.

The present invention aims to provide a solid cosmetic composition that meets all of these criteria, in particular that has a physiological pH, that is gentle on the skin and that contains a maximum number of ingredients of natural origin, and at the same time has excellent foaming ability, a beautiful appearance, in particular a shiny appearance and a homogeneous texture, that is pleasing for consumers.

Additional objectives of the invention are that this composition contains the lower possible number of ingredients, and that it is suitable for use on all skin types, including sensitive skin and atopic skin.

The aim of the invention is also to ensure that the consistency of this solid composition is such that it does not crumble when handled, or when stored over time, i.e., it is not too hard, but at the same time that it is not so soft and sticky that it would be impractical and unpleasant to use and difficult to manufacture by means of conventional devices for manufacturing solid compositions, in particular by extrusion.

### DISCLOSURE OF THE INVENTION

It has been discovered by the present inventors that such objectives are achieved by using a particular combination of surfactants, this combination combining a much higher degree of naturalness than the syndets commonly used in the field of cosmetics, as well as, unexpectedly, a particularly high foaming power when brought into contact with water, this foaming power expressing itself in particular in terms of rapid foam formation, abundance of foam and number of bubbles in the foam. This combination of surfactants also allows solid compositions to be formed that are both low in stickiness and of adequate hardness, with an attractive appearance, homogeneous texture and advantageous sensory characteristics.

Thus, according to a first aspect, the present invention proposes a solid cosmetic composition which contains:
a total of 30 to 65% by weight, relative to the total weight of the composition, of consistency agent(s),
at least one nonionic surfactant selected from alkyl glucosides in which the alkyl radical comprises 8 to 16 carbon atoms and/or at least one amphoteric surfactant other than amidopropyl betaines, advantageously of natural origin,
at least one anionic surfactant selected from isethionic acids and their cosmetically acceptable salts and sulfosuccinic acids and their cosmetically acceptable salts, the total content of anionic surfactant(s) selected from isethionic acids and their cosmetically acceptable salts and sulfosuccinic acids and their cosmetically acceptable salts being less than or equal to 20% by weight, relative to the total weight of the composition, and the total content of surfactants in the composition being between 10 and 50% by weight relative to the total weight of the composition.

Furthermore, this composition:
contains less than 8% by weight of water relative to the total weight of the composition,
and does not contain any gelling agent or contains less than 1% by weight of gelling agent(s), relative to the total weight of the composition.

A solid composition is defined in this description, in a conventional manner, as a composition in block form that does not flow under the effect of its own weight. This definition excludes, in particular, powdered forms.

More particularly, within the meaning of the present invention, a solid composition is defined as any composition that has a hardness (compressive strength) that is greater than or equal to 20 grams, at ambient temperature, i.e. at a temperature of between 2° and 25° C. This hardness can be measured by penetrating a cylindrical probe with a diameter of 0.8 cm into a matrix of the composition, to a depth of 8 mm at a speed of 1 mm/s, and measuring the penetration force of the probe. Any texture analysis device can be used for this purpose, in particular a TA.XT+ analyzer available from the Stable Micro Systems Company.

The solid cosmetic composition according to the invention is of the type intended to be used, in particular for hygiene and/or the care of skin and/or hair, directly in its solid form, as opposed to compositions in solid form intended to be dissolved in water before use so as to make a liquid composition, in which form they are intended to be used. Thus, unlike such compositions that are to be dissolved prior to use, the composition according to the invention does not contain, or contains very little, i.e. less than 1% by weight, preferably less than 0.5% by weight, preferably less than 0.1% by weight, and even more preferably less than 0.05% by weight, of gelling agents, relative to the total weight of the composition. In particular, the composition according to the invention does not contain or contains less than 1% by weight, preferably less than 0.5% by weight, preferably less than 0.1% by weight, and even more preferably less than 0.05% by weight, relative to the total weight of the composition, of gelling agents selected from gums, more particularly selected from xanthan gum, diutan gum, gellan gum, sclerotium gum, succinoglucan gum, gum arabic, carob gum, Tamarindus indica seed gum and Caesalpinia spinosa gum.

The composition according to the invention is furthermore preferably also free of substances capable of causing agitation when in contact with water, such as an effervescent base, in particular a combination of an organic acid and an alkaline and/or alkaline-earth carbonate or hydrogen carbonate, for example citric acid and sodium carbonate.

The solid cosmetic composition according to the invention may for example be packaged in the form of a bar, pebble, ball, baton or stick, optionally in a refillable packaging device, etc.

The composition according to the invention preferably also comprises at least one cosmetically acceptable excipient.

In the present description, the term “cosmetically acceptable” means that the salt or excipient is suitable for use in contact with human and animal cells, particularly skin or hair cells. Preferably, the excipient has a pleasant odor, color and/or feel, and does not cause unacceptable discomfort which could deter a user from using the composition according to the invention.

In the present description, the term “at least one” refers to a single compound or a mixture of several such compounds. Thus, as surfactants, the composition according to the invention may in particular contain:
a single nonionic surfactant selected from alkyl glucosides wherein the alkyl radical comprises 8 to 16 carbon atoms, or a plurality of such nonionic surfactants, and/or a single amphoteric surfactant other than amidopropyl betaines, or a plurality of such amphoteric surfactants,
and a single anionic surfactant selected from isethionic acids and their cosmetically acceptable salts and sulfosuccinic acids and their cosmetically acceptable salts, or a plurality of such anionic surfactants. Preferably, however, the composition according to the invention does not contain both an anionic surfactant selected from isethionic acids and their cosmetically acceptable salts and an anionic surfactant selected from sulfosuccinic acids and their cosmetically acceptable salts. Thus, the composition according to the invention preferably contains either one or several anionic surfactants selected from isethionic acids and their cosmetically acceptable salts, or one or several anionic surfactants selected from sulfosuccinic acids and their cosmetically acceptable salts.

Such a specific mixture of surfactants advantageously ensures that the composition has high foaming power when it comes into contact with water, as well as good foam quality. Without presuming the phenomena underlying the performance of the combination of surfactants according to the invention, it can be thought that they are due to a synergistic effect between the particular groups carried by these surfactants, in particular glucoside and/or amphoteric groups on the one hand, and isethionic acid/isethionate and/or sulfosuccinic acid/sulfosuccinate on the other hand, as well as to the particular chain length of the alkyl radical of the alkyl glucoside nonionic surfactant, which contributes to its foaming power.

In particular, it has been discovered by the present inventors that the partial replacement, in a solid cosmetic composition, of an anionic surfactant selected from isethionic acids and their cosmetically acceptable salts, by an alkyl glucoside in which the alkyl radical comprises 8 to 16 carbon atoms, in particular the replacement of 25 to 35% by weight of such an anionic surfactant with such an alkyl glucoside, does in no way impact the foaming power of the composition, and, on the contrary, even increases it in certain respects, while allowing the formation of compositions with a higher naturalness index. This is all the more unexpected given that non-ionic surfactants are known for their low foaming power. In particular, by combining an anionic surfactant selected from isethionic acids and their cosmetically acceptable salts with a nonionic surfactant selected from alkyl glucosides in which the alkyl radical comprises 8 to 16 carbon atoms, a high foaming power can be obtained for the composition with a particularly low total concentration of anionic surfactants relative to the concentrations recommended by the prior art, i.e. less than or equal to 20% by weight, and even less than or equal to 15% by weight, relative to the total weight of the composition.

In a general way, the solid cosmetic composition according to the invention preferably has a naturalness index, determined according to the calculation method of ISO 16128:2017, greater than or equal to approximately 85%, preferably greater than or equal to approximately 90%. All of its constituents are advantageously selected to achieve these values. Thus, the ingredients contained in the composition according to the invention are preferably of natural origin, thereby meeting the very strong demand for formulations containing ingredients of natural origin, which has been a trend in the cosmetics market for several years.

For this reason of naturalness, the composition according to the invention is preferably devoid of paraffin.

Furthermore, it preferably does not contain ingredients that are irritating to the skin, dry it out, might cause intolerance and/or allergenic. Thus, in addition to the fact that it is preferably devoid of amphoteric surfactant of the amidopropyl betaine class, preferably, the composition according to the invention is also devoid of anionic surfactant comprising at least one sulfate group, i.e. a —O—SO₃H or —O—SO₂—O— group, such as laureth sulfate or lauryl sulfate. Although sulfate surfactants have particularly good foaming properties, they are harsh on the skin.

Preferably, again in order to improve user tolerance, the composition according to the invention is also preferably free of microplastics of the acrylate or silicone class, and preferably free of compounds having an acrylate function and of silicone.

In particular embodiments of the invention, the composition does not contain any alkyl glucoside in which the alkyl radical has more than 16 carbon atoms, for example arachidyl glucoside. Such a substance, unlike the C8-C16 alkyl-containing alkyl glucosides recommended by the invention, imparts excessive hardness to the composition in particular. In particular embodiments of the invention, the composition does not contain any mixture of arachidyl glucoside and arachidyl alcohol.

It is also advantageously free of soap, i.e. substance obtained by saponification, such as substances with the INCI names sodium palmate, sodium cocoate, sodium olivate, sodium stearate, etc. The pH of the composition according to the invention is thus advantageously close to the physiological pH of the skin. It is preferably between 4.5 and 7, preferably between 5 and 6.5. The pH of the solid composition according to the invention can be measured by dissolving the composition in water, for example in a ratio of 1 g of composition to 100 g of water, and measuring the pH of the solution obtained.

The composition according to the invention further comprises 20% by weight or less, preferably 15% by weight or less, and preferably 10 to 15% by weight, relative to the total weight of the composition, of anionic surfactants selected from isethionic acids and their cosmetically acceptable salts and sulfosuccinic acids and their cosmetically acceptable salts. Its content of these anionic surfactants is therefore much lower than those of the solid cosmetic compositions of the prior art, which typically comprise nearly 40% by weight of such anionic surfactants.

The total surfactant content in the composition is furthermore between 10 and 50% by weight, preferably between 15 and 40% by weight, preferably between 15 and 35% by weight, and even more preferably between 20 and 30% by weight or between 15 and 25% by weight, relative to the total weight of the composition. In particular, it may be between 18 and 25% by weight, for example between 18 and 22% by weight, relative to the total weight of the composition. Such value ranges enable to obtain good foaming power for the composition and good quality of the foam, while also reducing the manufacturing costs of the composition.

The composition according to the invention comprises less than 8% by weight water, relative to the total weight of the composition. The water content of the composition according to the invention is preferably less than or equal to 7% by weight, preferably less than or equal to 6% by weight, in particular less than or equal to 5% by weight, relative to the total weight of the composition. In particular embodiments of the invention, the composition does not contain water, or only contains trace amounts of water. In variants of the invention, the composition comprises 0.5 to 4% by weight water, in particular from 1 to 3% by weight water, relative to the total weight of the composition.

The composition according to the invention may also have one or more of the characteristics described below, which may be used alone or in any technically effective combination.

In particularly preferred embodiments of the invention, the composition further contains at least one, i.e. one or more, anionic surfactant(s) selected from acyl glutamic acids and their cosmetically acceptable salts. The use of such an anionic surfactant, of natural origin, has the advantage of increasing the naturalness index of the composition, without reducing its foaming properties, in particular when it is incorporated into the composition as a partial replacement for the anionic surfactant(s) selected from isethionic acids and their cosmetically acceptable salts and sulfosuccinic acids and their cosmetically acceptable salts.

In particular embodiments of the invention, the total content of nonionic surfactant(s) selected from alkyl glucosides in which the alkyl radical comprises 8 to 16 carbon atoms, and/or amphoteric surfactant(s) other than amidopropyl betaines, in the composition is between 1 and 20% by weight, preferably between 2 and 15% by weight, more preferably between 2 and 10% by weight, and even more preferably between 3 and 8% by weight, relative to the total weight of the composition.

The total content of nonionic surfactant(s) in the composition is preferably between 10 and 35% by weight, in particular between 15 and 30% by weight, and preferably between 20 and 30% by weight, relative to the total weight of surfactants contained in the composition.

Preferably, all the nonionic surfactants contained in the composition according to the invention are alkyl glucosides in which the alkyl radical comprises 8 to 16 carbon atoms.

The total content of nonionic surfactant(s) selected from alkyl glucosides in which the alkyl radical comprises 8 to 16 carbon atoms in the composition is preferably between 1 and 20% by weight, preferably between 2 and 15% by weight, more preferably between 2 and 10% by weight, and even more preferably between 3 and 8% by weight, relative to the total weight of the composition.

The total content of amphoteric surfactant(s) other than amidopropyl betaines in the composition is preferably between 1 and 10% by weight, preferably between 1 and 5% by weight, relative to the total weight of surfactants contained in the composition.

The total content of anionic surfactant(s) selected from acyl glutamic acids and their cosmetically acceptable salts, when present in the composition, is preferably between 1 and 30% by weight, preferably between 1 and 20% by weight and more preferably between 5 and 15% by weight or between 3 and 8% by weight, relative to the total weight of the composition.

In particular embodiments of the invention, the total content of anionic surfactant(s) selected from acyl glutamic acids and their cosmetically acceptable salts, when present in the composition, is furthermore between 20 and 40% by weight, in particular between 20 and 30% by weight, relative to the total weight of surfactants contained in the composition.

When present in the composition according to the invention, the one or more anionic surfactants selected from acyl glutamic acids and their cosmetically acceptable salts are preferably selected from acyl glutamic acids the acyl group of which comprises a saturated or unsaturated, linear or branched hydrocarbon chain, with 8 to 30 carbon atoms, preferably with 10 to 20 carbon atoms and more preferably with 12 to 18 carbon atoms. The acyl group may, in particular, be selected from the lauroyl, myristoyl, palmitoyl, stearoyl, olivoyl, cocoyl and oleoyl groups. The cosmetically acceptable salt of the acyl glutamic acid can be selected from alkali metal salts, alkaline earth metal salts and ammonium salts. It is selected for example from sodium, potassium, lithium or magnesium salts, the sodium and potassium salts being particularly preferred in the context of the invention. The anionic surfactant(s) selected from acyl glutamic acids and their cosmetically acceptable salts may, for example, be selected in the group consisting of compounds with the INCI names: sodium cocoyl glutamate, disodium cocoyl glutamate, sodium myristoyl glutamate, sodium lauroyl glutamate, potassium lauroyl glutamate, potassium cocoyl glutamate and sodium stearoyl glutamate. Several anionic surfactants selected from acyl glutamic acids and their cosmetically acceptable salts, in particular those having the above characteristics, may be used in the composition according to the invention, which may, for example, contain any mixture of two or more of the compounds listed above. Preferably, the composition according to the invention comprises a single anionic surfactant selected from acyl glutamic acids and their cosmetically acceptable salts, this anionic surfactant then preferably having one or more of the characteristics set out above, or no such surfactant.

The nonionic surfactant(s) selected from alkyl glucosides in which the alkyl radical comprises 8 to 16 carbon atoms are preferably selected from compounds with the INCI names: octyl glucoside, lauryl glucoside and decyl glucoside. Several nonionic surfactant(s) selected from alkyl glucosides in which the alkyl radical comprises 8 to 16 carbon atoms may be used in the composition according to the invention, which may, for example, contain any mixture of two or more of the above compounds (octyl glucoside, lauryl glucoside and decyl glucoside). Preferably, the composition according to the invention comprises a single nonionic surfactant selected from alkyl glucosides in which the alkyl radical comprises 8 to 16 carbon atoms, this nonionic surfactant then preferably having one or more of the characteristics set out above, or no such surfactant.

The amphoteric surfactant(s) other than amidopropyl betaines may for example be selected from sodium caproamphoacetate, sodium caproamphopropionate, sodium capryloamphoacetate, sodium capryloamphopropionate, sodium cocoamphoacetate, sodium cocoamphopropionate, sodium isostearoamphoacetate, sodium isostearoamphopropionate, sodium lauroamphoacetate, sodium lauroamphopropionate, sodium oleoamphoacetate, sodium oleoamphopropionate, sodium stearoamphoacetate, stearoamphopropionate, sodium tallow amphoacetate, sodium wheatgerm amphoacetate, disodium caproamphodiacetate, disodium caproamphodipropionate, disodium capryloamphodiacetate, disodium cocoamphodiacetate, disodium isostearoamphodiacetate, disodium lauroamphodiacetate, disodium oleoamphodipropionate, disodium capryloamphodipropionate, disodium cocoamphodipropionate, disodium isostéaroamphodipropionate, disodium lauroamphodipropionate, disodium stearoamphodiacetate, disodium tallow amphodiacetate and disodium wheatgerm amphodiacetate.

Preferably, said at least one amphoteric surfactant other than amidopropyl betaines is selected from lauryl betaine, coco-betaine, sodium cocoamphoacetate and disodium cocoamphodiacetate.

Several amphoteric surfactant(s) other than amidopropyl betaines may be used in the composition according to the invention, which may, for example, contain any mixture of two or more of the above compounds, in particular two or more of: lauryl betaine, coco betaine, sodium cocoamphoacetate and disodium cocoamphodiacetate. Preferably, the composition according to the invention comprises a single amphoteric surfactant other than amidopropyl betaines, this amphoteric surfactant then preferably having one or more of the characteristics set out above, or no such surfactant.

In the class of amidopropyl betaines, excluded from the composition according to the invention, the following compounds may be mentioned in particular: behenamidopropyl betaine, capryl/capramidopropyl betaine, cocamidopropyl betaine, isostearamidopropyl betaine, lauramidopropyl betaine, myristamidopropyl betaine, stearamidopropyl betaine, undecyleneamidopropyl betaine, and amidopropyl betaines derived from almond, apricot, avocado, babassu, rapeseed, coconut oil, sunflower oil, oats, olive, palm oil, castor oil, soybean, shea butter or wheatgerm.

The anionic surfactant(s) selected from isethionic acids and their cosmetically acceptable salts are preferably selected from acyl isethionic acids and their cosmetically acceptable salts, such as substances with the INCI names: sodium cocoyl isethionate, sodium lauroyl isethionate, sodium cocoyl methyl isethionate and sodium lauroyl methyl isethionate. Several anionic surfactants selected from acyl isethionic acids and their cosmetically acceptable salts may be used in the composition according to the invention, which may, for example, contain any mixture of two or more of the above compounds. Preferably, the composition according to the invention comprises a single anionic surfactant selected from isethionic acids, preferably acyl isethionic acids, and their cosmetically acceptable salts, this anionic surfactant then preferably having one or more of the characteristics set out above, or no such surfactant.

The anionic surfactant(s) selected from sulfosuccinic acids and their cosmetically acceptable salts are preferably selected from alkyl sulfosuccinic acids and their cosmetically acceptable salts, such as the INCI named substance disodium lauryl sulfosuccinate. Several anionic surfactants selected from sulfosuccinic acids and their cosmetically acceptable salts may be used in the composition according to the invention. Preferably, the composition according to the invention comprises a single anionic surfactant selected from sulfosuccinic acids, preferably alkyl sulfosuccinic acids, and their cosmetically acceptable salts, this anionic surfactant then preferably having one or more of the characteristics set out above, or no such surfactant.

In general, the composition according to the invention preferably contains as few surfactants as possible. Thus, in particular embodiments of the invention, the composition does not comprise more than 6 surfactants, preferably not more than 5 surfactants, more preferably not more than 4 surfactants and preferably 3 or 2 surfactants. As indicated above, strong foaming properties can be obtained with two or three of the particular families of surfactants defined above, which is advantageous for minimizing the number of ingredients in the composition.

The composition according to the invention may contain other anionic surfactants than those defined above. Preferably, it does not comprise any anionic surfactants other than isethionic acids, isethionates, sulfosuccinic acids, sulfosuccinates, acyl glutamic acids and acyl glutamates. Similarly, the composition according to the invention may contain other nonionic surfactants than those defined above. Preferably, it does not comprise any nonionic surfactants other than alkyl glycosides whose alkyl radical comprises 8 to 16 carbon atoms.

The composition according to the invention may further comprise one or more cationic surfactants, for example when intended for the cosmetic treatment of hair, for a conditioner. Preferably, it does not contain any cationic surfactants of non-natural origin, and, more generally, it preferably does not contain any cationic surfactant at all.

In particular embodiments of the invention, the composition contains, as surfactants, only:
one or several, preferably a single, nonionic surfactant(s) selected from alkyl glucosides in which the alkyl radical comprises 8 to 16 carbon atoms, or one or several amphoteric surfactant(s) other than amidopropyl betaines,
one or several, preferably a single, anionic surfactant(s) selected from isethionic acids, preferably acyl isethionic acids, and their cosmetically acceptable salts and sulfosuccinic acids, preferably alkyl sulfosuccinic acids, and their cosmetically acceptable salts,
and, optionally, one or several, preferably a single, anionic surfactant(s) selected from acyl glutamic acids and their cosmetically acceptable salts.

Preferably, the composition contains, as surfactants, only:
one or several, preferably a single, nonionic surfactant(s) selected from alkyl glucosides in which the alkyl radical comprises 8 to 16 carbon atoms, preferably in a content of between 2 and 10% by weight, for example between 3 and 8% by weight, relative to the total weight of the composition,
one or several, preferably a single, anionic surfactant(s) selected from isethionic acids, preferably acyl isethionic acids, and their cosmetically acceptable salts, preferably in a content of between 10 and 20% by weight, for example between 10 and 15% by weight, relative to the total weight of the composition,
and, optionally, one or several, preferably a single, anionic surfactant(s) selected from acyl glutamic acids and their cosmetically acceptable salts, preferably in a content of between 1 and 20% by weight, for example between 3 and 8% by weight, relative to the total weight of the composition.

In such embodiments, more preferably, the respective contents of each of said types of surfactants, relative to the total content of surfactants in the composition, expressed as % by weight relative to the total weight of surfactants in the composition, are as follows:
20 to 30% nonionic surfactant(s) selected from alkyl glucosides in which the alkyl radical comprises 8 to 16 carbon atoms, and 70 to 80% anionic surfactant(s) selected from isethionic acids, preferably acyl isethionic acids, and their cosmetically acceptable salts,
or 20 to 30% nonionic surfactant(s) selected from alkyl glucosides in which the alkyl radical comprises 8 to 16 carbon atoms, 40 to 60% anionic surfactant(s) selected from isethionic acids, preferably acyl isethionic acids, and their cosmetically acceptable salts, and 20 to 30% anionic surfactant(s) selected from acyl glutamic acids and their cosmetically acceptable salts.

The composition according to the invention comprises at least one fatty phase consistency agent, in a content of between 30 and 65% by weight, preferably between 30 and 60% by weight, preferably between 35 and 55% by weight, relative to the total weight of the composition. Such a concentration of consistency agent(s) in the composition, in combination with the filler(s) that may also be contained therein, as mentioned below, advantageously ensures optimum hardness and stickiness characteristics of the composition, in particular, a hardness that is low enough so that the composition does not crumble when handled by the user and high enough for the composition to have a consistency that allows it to be easily handled, and a stickiness that is strong enough for the composition to have good cohesion, while being low enough not to diminish its practical and pleasant characteristics in use.

The composition according to the invention may in particular contain one or more consistency agent(s) selected from fatty alcohols, preferably C16-C22 fatty alcohols, fatty acids, in particular C16-C18 fatty acids, hydrogenated vegetable oils and hydrogenated castor oil.

Preferably, it contains at least one consistency agent selected from C16-C22 fatty alcohols, such as cetyl alcohol, stearyl alcohol, arachidyl alcohol, and/or behenic alcohol.

In particular embodiments of the invention, when the composition comprises, as consistency agents, at least one or more fatty alcohols and one or more fatty acids, then the total fatty alcohol(s) content therein is higher, preferably at least 4 times higher, preferably at least 5 times higher and even 6 times higher, than the total fatty acid(s) content. Such a characteristic advantageously ensures that the composition has good qualitative properties, in particular a homogeneous texture, a nice smooth and shiny appearance, and a good consistency.

In particular embodiments, the composition according to the invention, when it contains one or more fatty acid(s) as consistency agent(s), then also contains at least one fatty alcohol or a hydrogenated vegetable oil or hydrogenated castor oil.

In preferred embodiments of the invention, at least one of the consistency agents is contained in the composition in an individual content that is greater than 15% by weight, preferably greater than 20% by weight, more preferably greater than or equal to 30% by weight, preferably greater than or equal to 35% by weight, and in particular greater than or equal to 40% by weight, relative to the total weight of the composition. This consistency agent is then preferably a C16-C22 fatty alcohol, such as cetyl alcohol, stearyl alcohol, arachidyl alcohol or behenic alcohol.

The content of this consistency agent in the composition may for example be greater than 15% by weight and less than or equal to 65% by weight, preferably greater than 20% by weight and less than or equal to 65% by weight, or even between 30 and 65% by weight, preferably between 30 and 60% by weight, and preferably between 35 and 55% by weight, relative to the total weight of the composition.

In particularly preferred embodiments of the invention, at least one consistency agent is present in the composition in a content that is higher than the total content in the composition of the anionic surfactant(s) selected from isethionic acids and their cosmetically acceptable salts and sulfosuccinic acids and their cosmetically acceptable salts.

Thus, in embodiments of the invention in which the total content of anionic surfactant(s) selected from isethionic acids and their cosmetically acceptable salts and sulfosuccinic acids and their cosmetically acceptable salts is less than or equal to 20% by weight relative to the total weight of the composition, it is advantageous according to the invention that the composition contains at least one consistency agent in an individual content greater than 20% by weight relative to the weight of the composition, this consistency agent being then preferably a C16-C22 fatty alcohol.

In the preferred embodiments of the invention in which the total content of anionic surfactant(s) selected from isethionic acids and their cosmetically acceptable salts and sulfosuccinic acids and their cosmetically acceptable salts is less than or equal to 15% by weight relative to the total weight of the composition, then it is sufficient for the composition to contain at least one consistency agent in an individual content greater than 15% by weight relative to the weight of the composition. Again, this consistency agent is then preferably a C16-C22 fatty alcohol.

The composition according to the invention may further comprise one or more emulsifiers, such as for example glycol palmitate (INCI name) or the products marketed under the names Montanov® 68 (Cetearyl Alcohol (and) Cetearyl Glucoside) and Montanov® 82 (Cetearyl Alcohol (and) Coco-Glucoside). Otherwise, it may be devoid of them. Preferably, it is devoid of emulsifier formed from a mixture with the INCI name “arachidyl alcohol, behenyl alcohol, arachidyl glucoside”, such as the mixture marketed under the name Montanov® 202.

In particular embodiments of the invention, the composition contains one or several cosmetically acceptable excipient(s). Each of these excipients is preferably selected from fillers, emollients, in particular oils, conditioning agents, stabilizers, fragrances, colorants and pigments.

In particular embodiments of the invention, the composition comprises, as a cosmetically acceptable excipient, at least one filler, which may be of either mineral or organic origin.

Mineral-based fillers that can be used in the composition according to the invention include clays, ceramic beads, calcium carbonate, titanium oxides, kaolin and aluminum silicate. Organic-based fillers include starches, maltodextrin, celluloses such as cellulose beads, microcrystalline cellulose, sodium carbonate, nylon microspheres, micronized or non-micronized plant powders such as fruit powders or bamboo powders, and rice husk powder, and mixtures thereof.

Preferably, the composition according to the invention contains at least one filler selected from kaolin, maltodextrin, starches, celluloses, calcium carbonate, and mixtures thereof.

The starches that can be used in the present invention may be derived from a plant source such as cereals, tubers, roots, vegetables, and fruits. These may include, for example, corn starch, potato starch, pea starch, rice starch, oat starch, barley starch, tapioca starch, wheat starch, cassava starch or sorghum. These starches may or may not be modified. A modified starch is a starch that has been modified by processes known to the person skilled in the art, such as, for example, esterification, etherification, oxidation, acid hydrolysis, crosslinking or enzymatic conversion. As cellulose, for example, microcrystalline cellulose as sold under the name Vivapur® CS 9 FM may be used. Kaolin sold under the name ImerCare® Opaque by the company Imerys may be used, for example.

The total amount of filler(s) in the composition according to the invention is preferably between 5 and 25% by weight, preferably between 5 and 20% by weight, for example between 15 and 20% by weight, relative to the total weight of the composition.

Preferably, at least one of the fillers contained in the composition according to the invention is present in a content greater than 15% by weight relative to the total weight of the composition.

Thus, preferably, the total content of consistency agent(s) in the composition, in particular when the composition contains only one or more fatty alcohol(s) as consistency agent(s), or when it mainly contains one or more fatty alcohol(s) as consistency agent(s), is greater than the total content of filler(s) in the composition.

Furthermore, when the composition contains one or more oils, the total content of filler(s) is preferably greater than the total content of oil(s).

In particular embodiments of the invention, the oil content of the composition is less than 5% by weight, preferably less than 4% by weight and preferentially less than 3% by weight, relative to the total weight of the composition.

The composition according to the invention may contain one or more emollients, in particular selected from vegetable oils, such as sunflower oil, sweet almond oil, argan oil or jojoba oil, butters, such as shea butter, for example as sold under the name Lipex SheaLight® or Cetiol® SB 45, glycerin, lauroyl lysine, for example as sold under the name Amihope® LL.

The emollient content of the composition according to the invention is, for example, between 2 and 20% by weight, preferably between 2 and 15% by weight, and in particular between 2 and 10% by weight, relative to the total weight of the composition.

The composition according to the invention may also contain one or more pH adjusters. It preferably does not contain any pH adjuster, in particular of the NaOH, citric acid, lactic acid, etc., type.

In particular embodiments of the invention, the composition contains one or more conditioning agent(s), preferably not belonging to the category of cationic surfactants. Such agents provide beneficial effects for both hair and skin. Each of these conditioning agents may in particular be selected from isoamyl laurate, lauroyl lysine, erythritol, glyceryl oleate, for example as sold under the name DUB® OG, polyglyceryl-4 caprate, the combination of chitosan and mandelic acid, or any mixture thereof. The total amount of conditioning agent(s) in the composition is preferably between 0.01 and 3% by weight, preferably between 0.05 and 2% by weight, and preferably between 0.1 and 2% by weight, relative to the total weight of the composition.

The composition according to the invention may further contain one or more conditioning agent(s) selected from guar and derivatives thereof, such as hydroxypropyl guar, in a total amount of between 0.01 and 3% by weight, preferably between 0.05 and 2% by weight, and preferably between 0.1 and 2% by weight, relative to the total weight of the composition.

Optionally, the composition according to the invention may additionally contain one or more stabilizer(s), such as chelating agents, for example tetrasodium glutamate diacetate, as sold under the name Dissolvine® GL-47-S, vitamin E, or tocopherol acetate, as sold under the name Covi-ox® T-90.

The composition according to the invention may contain one or more cosmetically active agents, in particular by topical route, for example selected from hydrating agents, antioxidants, vitamins, etc.

The content of cosmetically active agent(s) in the composition according to the invention may, for example, be between 0.05 and 3% by weight relative to the total weight of the composition.

In particular embodiments of the invention, the composition comprises one or more cosmetically active agent(s), each being preferably selected from hyaluronic acid, oat sap, helychrysum extracts, helychrysum essential oils, limnanthe oil, in particular Limnanthes alba seed oil, oat seedlings, flower acids, such as the product marketed under the name Acides de Fleurs®, Garcinia extracts, milk thistle oil, niacinamide, edelweiss extracts, such as the product marketed under the name Alpaflor® Edelweiss, quinquina extracts, caffeine, peony extracts, mango butter and citron extracts, without this list being exhaustive in any way.

The composition according to the invention may also contain an exfoliating agent, which will enable exfoliation of the skin (including the scalp) by mechanical and/or chemical action, the exfoliating agents acting both by mechanical and chemical action being particularly preferred in the context of the invention. The exfoliating agent may be selected from agents of mineral, plant or organic origin. Thus, for example, it may be used: polyethylene beads or powder, nylon particles, polyvinyl chloride powder, pumice stone (INCI name: pumice), walnut shells, sawdust, wood flour, cork flour, glass beads, alumina (aluminum oxide) (INCI name: Alumina), sugar crystals, beads that melt upon application to the skin, such as, for example, mannitol and cellulose-based spheres, agar-based capsules, jojoba ester-based spheres, fruit kernel powders, for example apricot kernel, and mixtures thereof. The exfoliating agent(s) used are preferably not soluble in water.

In preferred embodiments of the invention, the composition does not contain polyethylene glycol (PEG).

Furthermore, it is also preferably free of aluminum and magnesium silicate, and, more generally, silica.

The composition according to the invention, in its desired form, can be prepared using any conventional method per se for the preparation of solid cosmetic compositions. Particularly preferred preparation methods in the context of the invention are extrusion and hot casting methods.

A method for preparing a composition according to the invention by extrusion comprises preparing a bondillon containing the surfactants of the composition, the consistency agent(s) and the excipient(s), to which the other possible ingredients, in particular active agent(s) and/or the fragrance, are then added. The mixture is then extruded through different grids to homogenize and refine it. Once the mixture is homogeneous, it forms a lump that is cut and tapped to obtain a solid composition of the desired shape.

A method for preparing a composition according to the invention by hot casting comprises mixing the ingredients of the composition and heating the mixture obtained to a temperature of about 75-80° C. The homogenized hot mixture is then cast into molds of the desired shape, and then allowed to cool.

Both of these preparation methods are quick and easy to implement.

Another aspect of the invention relates to the non-therapeutic cosmetic use of a solid cosmetic composition according to the invention for washing and/or caring (including exfoliating) for the skin, in particular the skin of the body and/or face and/or scalp, and/or for the hair, of an individual, preferably by topical application.

This use preferably comprises successive steps consisting of:
wetting the composition with water,
developing foam, in particular by rubbing the wetted surface of the composition,
applying this foam to the skin and/or hair, the latter having preferably been made wet beforehand,
and rinsing, preferably with water, optionally after a resting time and/or a massage.

These steps can, for example, be performed once or twice per day, for example morning and evening, or as often as necessary.

The solid cosmetic composition according to the invention can be used, in particular, for washing and/or caring for sensitive or atopic skin.

In the present description, sensitive skin refers to skin that is prone to irritation or allergic reactions due to various internal and external non-immunological factors. The term atopic skin refers to skin affected by an inflammatory condition characterized by dryness of the skin associated with itching such as eczema or atopic dermatitis, for example.

The invention also relates to a non-therapeutic cosmetic treatment method for the hygiene and/or care of the skin and/or hair of an individual, by means of a solid cosmetic composition according to the invention. This method comprises successive steps consisting of:
wetting the composition with water,
developing foam,
applying the foam to the skin and/or hair,
and rinsing, preferably with water.

This method can respond to any feature, or combination of features, described above with reference to the cosmetic use of the composition according to the invention.

### EXAMPLES

The features and advantages of the invention will become more apparent in light of the examples of implementations below, provided purely by way of illustrative and non-limiting examples of the invention.

### 1/Examples of Compositions

Solid compositions according to the invention, C1, C2 and C3, intended for cosmetic treatment, for hygiene and care, of the skin of the body and the face, meet the characteristics shown in Table 1 below.

TABLE 1
Examples of compositions according to the invention
for hygiene and skin care of the body and face

| | C1 | C2 | C3 |
|---|---|---|---|
| Ingredients (INCI name) | % w/w | % w/w | % w/w |
| Sodium cocoyl isethionate | 10 | 10 | 15 |
| Decyl glucoside | 5 | 5 | 5 |
| Sodium myristoyl glutamate | 5 | 5 | — |
| Behenyl alcohol | 45 | 43 | 45 |
| Stearic acid/palmitic acid | 7 | 7 | 7 |
| Corn starch | 20 | 20 | 20 |
| Polyglyceryl-4 caprate | — | 2 | — |
| Sunflower oil | 2 | 2 | 2 |
| Shea butter | 1 | 1 | 1 |
| Water | 5 | 5 | 5 |

Solid compositions not in accordance with the invention, Comp4, Comp5, Comp6, Comp7 and Comp8, intended for cosmetic treatment, for hygiene and care of the skin of the body and face, meet the characteristics listed in Table 2 below.

TABLE 2
Examples of compositions not in accordance with the invention

| | Comp4 | Comp5 | Comp6 | Comp7 | Comp8 |
|---|---|---|---|---|---|
| Ingredients (INCI name) | % w/w | % w/w | % w/w | % w/w | % w/w |
| Sodium cocoyl | 10 | 20 | 10 | 10 | 20 |
| isethionate | | | | | |
| Decyl glucoside | — | — | — | — | — |
| Sodium myristoyl | 10 | — | 5 | 10 | — |
| glutamate | | | | | |
| Behenyl alcohol | 45 | 45 | 43 | 43 | 43 |
| Stearic acid/palmitic | 7 | 7 | 7 | 7 | 7 |
| acid | | | | | |
| Corn starch | 20 | 20 | 20 | 20 | 20 |
| Polyglyceryl-4 caprate | — | — | 2 | 2 | 2 |
| Sunflower oil | 2 | 2 | 2 | 2 | 2 |
| Shea butter | 1 | 1 | 1 | 1 | 1 |
| Water | 5 | 5 | — | 5 | 5 |
| Montanov ® 202 | — | — | 10 | — | — |
| (behenyl alcohol/ | | | | | |
| arachidyl alcohol/ | | | | | |
| arachidyl glucoside) | | | | | |

### 2/Evaluation of Characteristics of the Compositions

The compositions of example 1 are evaluated according to the following criteria:
foaming power (quantity/quality): foam height, foam creaminess, foam softness,
good tolerance, physiological pH, hypoallergenic, softer and gentler on the skin,
stability over time,
organoleptic characteristics (color, odor)
hardness, as measured by a texture analyzer, the composition must have sufficient consistency so as not to crumble but not be to too soft,
stickiness, as measured by a texture analyzer, the composition must have a sufficient level of stickiness so as not to crumble, but not too high so as to be pleasant to use and able to be prepared in good conditions, in particular in an extrusion device,
pH: the measurement is made using a pH meter after diluting 1 g of the composition in 100 g of water, the objective being to check that the pH corresponds to the physiological pH of the skin.

For this purpose, the following operating protocols are implemented.

A TA.XTplus texture analyzer with a penetration and withdrawal rate of 1 mm/s, a trigger force of 5 g, a distance of 8 mm and a P/P2N needle is used for the evaluation of hardness and stickiness. The composition is fixed on the table of the texturometer, and the following are measured: the needle penetration force (hardness measurement) and the needle withdrawal force (evaluation of the stickiness). The results are given in grams.

For the evaluation of the foam height, a 250-ml graduated measuring cylinder, of outer diameter 38.9 cm and inner diameter 36 cm, with a stopper, is used. 0.5 g of composition is placed in the cylinder and demineralized water is added to make up to 50 ml. The cylinder is stoppered then vigorously shaken 10 times. The foam height is read and expressed in mm.

The approximate naturalness index of each of the compositions is also determined, according to the calculation method of ISO 16128:2017.

The results obtained are shown in Table 3 below.

TABLE 3
Characteristics of the compositions

| Composition | Naturalness index | pH | Foam height |
|---|---|---|---|
| C1 | +++ | 5.84 | +++ |
| C2 | +++ | 5.89 | +++ |
| C3 | ++ | 6.32 | +++ |
| Comp4 | +++ | 5.78 | + |
| Comp5 | + | 4.96 | +++ |
| Comp6 | +++ | 5.75 | + |
| Comp7 | +++ | 5.76 | + |
| Comp8 | + | 4.98 | ++ |

Naturalness index: + indicates an index less than or equal to 80%; ++ indicates an index greater than 80% and less than 90%; and +++ indicates an index greater than or equal to 90%
Foam height: + indicates height less than or equal to 60 mm; ++ indicates height greater than 60 mm and less than 95 mm; and +++ indicates height greater than or equal to 95 mm
The comparative compositions Comp5 and Comp8, comprising only sodium cocoyl isethionate as the surfactant, have very good foam height, as expected, but at the expense of the total naturalness of these compositions, which is less than 80%, with the same other ingredients. Furthermore, the pH of these compositions appears to be quite low (close to the lower range of the physiological pH of the skin).
Furthermore, it is observed that the compositions according to the invention C1, C2 and C3 all have a foam height:
as large, or even larger, than that of the comparative compositions Comp5 and Comp8, based on sodium cocoyl isethionate alone as a surfactant and of otherwise comparable composition, with an equal total surfactant content,
and significantly higher than those of the comparative compositions Comp4, Comp6, and Comp7, which have comparable naturalness indices, of 85% or more.

The association of decyl glucoside with sodium cocoyl isethionate thus makes it possible to increase the naturalness index of the composition, without being detrimental to the foaming power of the composition. An even higher naturalness index, greater than or equal to 90%, can be obtained without compromising the foaming power, by incorporating sodium myristoyl glutamate into the composition, in replacement of part of the sodium cocoyl isethionate.

The presence of a conditioner such as polyglyceryl-4 caprate in the composition according to invention C2 is advantageously not detrimental to the foaming power of the composition. On the contrary, when such an ingredient is contained in the comparative compositions, it adversely impacts the foaming power (decrease of 10 mm in the foam height for composition Comp8 relative to Comp5, and for composition Comp7 relative to Comp4). Thus, a conditioning agent may be included in the composition according to the invention, without affecting its foaming power. Such a characteristic is particularly advantageous, as it allows compositions with improved sensory properties to be created without any loss of foaming efficacy.

The hardness and stickiness are entirely satisfactory for all compositions according to the invention C1 to C3. These compositions have a hardness and a stickiness that ensure that they do not crumble and are neither too soft nor too sticky, so that they are practical and pleasant to use and can be easily shaped, in particular in an extrusion device. They also have an attractive appearance and a homogeneous texture.

### 3/Influence of the Consistency Agent on the Foaming Power

A composition according to the invention C9 is described in Table 4 below. This composition differs from the composition C1 according to the invention described above, the composition of which is reminded in Table 4, only in that the ratio of fatty alcohol(s) (INCI name “Behenyl alcohol”)/fatty acid(s) (INCI name: “Stearic acid/palmitic acid”) (consistency agents) is inverted.

The foam height obtained for each of these compositions is also shown in Table 4.

TABLE 4
Foam height obtained for compositions according to the invention
differing in the combination of consistency agents

| Composition | C1 | C9 |
|---|---|---|
| Ingredients (INCI name) | % w/w | % w/w |
| Sodium cocoyl | 10 | 10 |
| isethionate | | |
| Decyl glucoside | 5 | 5 |
| Sodium myristoyl | 5 | 5 |
| glutamate | | |
| Behenyl alcohol | 45 | 7 |
| Stearic acid/palmitic acid | 7 | 45 |
| Corn starch | 20 | 20 |
| Sunflower oil | 2 | 2 |
| Shea butter | 1 | 1 |
| Water | 5 | 5 |
| Foam height | +++ | ++ |

Foam height: ++ indicates height greater than 60 mm and less than 95 mm; and +++ indicates height greater than or equal to 95 mm
As can be seen, the foaming power is optimized by the presence of a fatty alcohol in majority amount among the consistency agents. When the amount of fatty alcohol is greater than the amount of fatty acid, as in composition C1, the foaming power is particularly high. When the amount of fatty alcohol is less than the amount of fatty acid, as in composition C9, the foaming power remains satisfactory, although lower.

### 4/Examples of Hair Compositions According to the Invention

Solid cosmetic compositions according to the invention, C11, C12 and C13, particularly suitable for the cosmetic treatment of hair, are described in Table 5. This table also shows the foam height measured for each according to the protocol of Example 2.

TABLE 5
Solid hair cosmetic compositions according to the invention

| Composition | C11 | C12 | C13 |
|---|---|---|---|
| Ingredients (INCI name) | % w/w | % w/w | % w/w |
| Sodium cocoyl isethionate | 10 | 10 | 15 |
| Decyl glucoside | 3-8 | 3-8 | 2-10 |
| Sodium myristoyl glutamate | 5-15 | 5 | — |
| Behenyl alcohol | 44 | 40-50 | 40-50 |
| Stearic acid/palmitic acid | 7 | 1-10 | 1-10 |
| Corn starch | 19 | 15-25 | 15-25 |
| Glyceryl oleate | 0.5 | — | — |
| Hydroxypropyl guar | — | 0.3 | — |
| Isoamyl laurate | — | — | 1.8 |
| Quinine extract | 1.5 | 1.5 | 1.5 |
| Sunflower oil | 2 | 2 | 2 |
| Shea butter | 1 | 1 | 1 |
| Water | Qs 100 | Qs 100 | Qs 100 |
| Foam height | +++ | +++ | +++ |

+++ indicates a foam height greater than or equal to 95 mm
All three compositions combine a high degree of naturalness and high foaming power.

### 5/Sensory Profiles of a Composition According to the Invention and of a Commercial Syndet Based on Isethionate

Two compositions are tested in this example: a composition C10 according to the invention for hygiene and skin care of the body and face, and a commercially available solid composition Comp14, not in accordance with the invention, a solid hibiscus facial cleanser for dry and sensitive skin. The purpose of this test is to compare the sensory characteristics of these 2 compositions.

The composition C10 according to the invention is described in Table 6 below:

TABLE 6
Composition C14 according to the invention

| Ingredients (INCI name) | % w/w |
|---|---|
| Sodium cocoyl isethionate | 10 |
| Decyl glucoside | 5 |
| Sodium myristoyl glutamate | 5 |
| Behenyl alcohol | 45 |
| Stearic acid/palmitic acid | 7 |
| Corn starch | 20 |
| Sunflower oil | 2 |
| Shea butter | 1 |
| Silybum marianum oil | 1 |
| Fragrance | qs |
| Water | Qs 100 |

For the commercial solid composition Comp14, not in accordance with the invention, the list of ingredients as indicated on the packaging is as follows (INCI names):
sodium cocoyl isethionate, stearic acid, palmitic acid, Cocos nucifera oil, Avena sativa kernel flour, aqua, Hibiscus sabdariffa flower powder, Helianthus annuus seed oil, decyl glucoside, Lavandula hybrida oil, Arnica montana flower extract, tocopherol, geraniol, limonene, linalool.
Sodium cocoyl isethionate is the main ingredient in this composition, its weight content in the composition being higher than that of the consistency agents stearic acid and palmitic acid.

The evaluation of the sensory profile of the compositions is carried out in monadic by 15 volunteers trained in sensory techniques for topical products, and aims to qualify:
firstly, the foaming power when applying the product (phase 1), (the product is then rinsed),
and, secondly, the aspect of the skin 15 minutes after rinsing (phase 2).

The tests are carried out in a room where the lighting, temperature and humidity are monitored. The rinsing is carried out at a steady flow rate with water at 37° C.

Different criteria are evaluated and are detailed in Table 7 below.

TABLE 7
Description of the criteria evaluated

| | |
|---|---|
| Criteria | Description |

Phase 1: Application

| | |
|---|---|
| Foam start | Fastest foaming product |
| Amount of foam | Plenty of foam |
| Amount of bubbles | Number of bubbles |
| Bubble sizes | Bubble thickness |
| Smoothness | Most creamy foam |
| Oily | Like cooking oil between the fingers |
| Squeaking | Intermittent sticking of the product between fingers |

Phase 2: 15 minutes after rinsing, on dry skin

| | |
|---|---|
| Tension | Tightness sensation |
| Brightness | Skin's ability to reflect |
| Resistant | Slows down the passage of the inside of the hand |
| | on the application area |
| Film-forming | Persistence of a film on the skin |
| Powder Touch | Like peach skin |
| Flexibility | Skin elasticity |

A computer questionnaire presents the unstructured analog measurement scales ranging from 0 to 100, for each of the criteria on the computer screen.

The results of the evaluation are summarized in Table 8 below.

TABLE 8
Sensory evaluation results

| | | |
|---|---|---|
| Criterion | C10 | Comp14 |

Phase 1: Application:

| | | |
|---|---|---|
| Foam start | 69** | 34 |
| Amount of foam | 49** | 15 |
| Amount of bubbles | 27** | 7 |
| Bubble sizes | 22 | 6 |
| Smoothness | 63 | 55 |
| Oily | 25 | 27 |
| Squeaking | 14 | 8 |

Phase 2: 15 minutes after rinsing, on dry skin

| | | |
|---|---|---|
| Tension | 14 | 15 |
| Brightness | 10 | 9 |
| Resistant | 3 | 1 |
| Film-forming | 9 | 5 |
| Powder Touch | 36 | 39 |
| Flexibility | 48 | 47 |

**indicates a significant difference
It is observed that significant differences (**) are obtained when applying the compositions. The composition according to the invention C10 allows for faster foam formation, the foam is more abundant and contains more bubbles, and significantly more compared to the commercially available composition Comp14.

It should be noted that the sensory analysis results for these two compositions are similar in terms of skin aspect characteristics 15 minutes after rinsing off the developed composition. Thus, the improvements in terms of foam were not obtained at the expense of the other sensory criteria presented above.

1. A solid cosmetic composition, which contains:
30 to 65% by weight of consistency agent(s), relative to the total weight of the composition,
at least one nonionic surfactant selected in the group consisting of alkyl glucosides in which the alkyl radical comprises 8 to 16 carbon atoms and/or at least one amphoteric surfactant other than amidopropyl betaines,
at least one anionic surfactant selected in the group consisting of isethionic acids and their cosmetically acceptable salts and sulfosuccinic acids and their cosmetically acceptable salts, the total content of anionic surfactant(s) selected in the group consisting of isethionic acids and their cosmetically acceptable salts and sulfosuccinic acids and their cosmetically acceptable salts being less than or equal to 20% by weight, relative to the total weight of the composition,
and the total surfactant content in said composition being between 10 and 50% by weight relative to the total weight of the composition,
less than 8% by weight of water relative to the total weight of the composition,
and which does not contain any gelling agent or contains less than 1% by weight of gelling agent(s), relative to the total weight of the composition.

2. The composition according to claim 1, wherein at least one of said consistency agents is contained in the composition in an amount greater than 20% by weight, relative to the total weight of the composition.

3. The composition according to claim 1, further containing at least one anionic surfactant selected in the group consisting of acyl glutamic acids and their cosmetically acceptable salts.

4. The composition according to claim 3, wherein the acyl group of said at least one anionic surfactant selected in the group consisting of acyl glutamic acids and their cosmetically acceptable salts is a lauroyl, myristoyl, palmitoyl, stearoyl, olivoyl, cocoyl or oleoyl group.

5. The composition according to claim 1, wherein said at least one nonionic surfactant is octyl glucoside, lauryl glucoside or decyl glucoside.

6. The composition according to claim 1, wherein said at least one amphoteric surfactant other than amidopropyl betaines is coco-betaine, lauryl betaine, sodium cocoamphoacetate or disodium cocoamphodiacetate.

7. The composition according to claim 1, containing, as surfactants, only:
one or several nonionic surfactant(s) selected in the group consisting of alkyl glucosides in which the alkyl radical comprises 8 to 16 carbon atoms,
one or several anionic surfactant(s) selected in the group consisting of isethionic acids and their cosmetically acceptable salts,
and, optionally, one or several anionic surfactant(s) selected in the group consisting of acyl glutamic acids and their cosmetically acceptable salts.

8. The composition according to claim 1, wherein the content of nonionic surfactant(s) selected in the group consisting of alkyl glucosides wherein the alkyl radical comprises 8 to 16 carbon atoms and/or amphoteric surfactant(s) other than amidopropyl betaines is between 1 and 20% by weight relative to the total weight of the composition.

9. The composition according to claim 1, containing one or more consistency agents selected in the group consisting of fatty alcohols, fatty acids, and hydrogenated castor oil.

10. The composition according to claim 9, containing at least one consistency agent selected in the group consisting of C16-C22 fatty alcohols.

11. The composition according to claim 1, devoid of anionic surfactant comprising at least one sulfate group and/or of paraffin.

12. The composition according to claim 1, containing at least one cosmetically acceptable excipient selected in the group consisting of fillers, emollients, conditioning agents, stabilizers, fragrances, pigments, colorants and mixtures thereof.

13. The composition according to claim 12, comprising at least one filler selected in the group consisting of kaolin, maltodextrin, starches, celluloses, calcium carbonate, and mixtures thereof.

14. The composition according to claim 12, comprising a total amount of filler(s) comprised between 5 and 25% by weight relative to the total weight of the composition.

15. The composition according to claim 1, further containing an exfoliating agent.

16. The composition according to claim 1, comprising at least one cosmetically active agent selected in the group consisting of hyaluronic acid, oat sap, helychrysum extracts, helychrysum essential oils, limnanthes oil, oat seedlings, flower acids, Garcinia extracts, milk thistle oil, niacinamide, edelweiss extracts, cinchona extracts, caffeine, peony extracts, mango butter and citron extracts.

17. The composition according to claim 1, packaged in the form of a bar, pebble, stick or ball.

18. A cosmetic method for washing and/or caring for the skin and/or hair, comprising applying to said skin and/or hair a composition according to claim 1.

19. The method according to claim 18, for washing and/or caring for sensitive skin.

20. The method of claim 18, comprising successive steps consisting of:
wetting said composition with water,
developing foam,
applying said foam to the skin and/or hair,
and rinsing.
